(19) United States
(12) Patent Application Publication
(10) Pub. No.: US 2022/0135502 A1
(43) Pub. Date: May 5, 2022

(54) AN ADSORBENT FOR SEPARATING ORGANOCHLORIDE COMPOUND FROM LIQUID HYDROCARBON AND A PROCESS THEREOF

(71) Applicant: PTT GLOBAL CHEMICAL PUBLIC COMPANY LIMITED, Bangkok (TH)

(72) Inventors: Yanee VONGACHARIYA, Bangkok (TH); Sitthiphong PENGPANICH, Bangkok (TH); Metta CHAREONPANICH, Nonthaburi (TH); Waleeporn DONPHAI, Samut Sakhon (TH); Thongthai WITOON, Chachoengsao (TH); Preeyaporn RUENGRUNG, Pathum Thani (TH)

(73) Assignee: PTT GLOBAL CHEMICAL PUBLIC COMPANY LIMITED, Bangkok (TH)

(21) Appl. No.: 17/605,461

(22) PCT Filed: Apr. 23, 2020
(86) PCT No.: PCT/IB2020/053840

§ 371 Date: Oct. 21, 2021

(30) Foreign Application Priority Data
Apr. 23, 2019 (TH) 1901002478

(51) Int. Cl. C07C 17/389; B01J 20/16; B01J 20/28; B01D 15/08

(52) U.S. Cl. CPC C07C 17/389; B01J 20/16; B01J 20/2808; B01J 2220/42; B01J 20/28085; B01J 20/28092; B01D 15/08; B01J 20/28083

(57) ABSTRACT

The present invention relates to the adsorbent for separating organochloride compound from liquid hydrocarbon and a process thereof, wherein said adsorbent is the silica and aluminosilicate composite having infiltrate structure subjected to the modification of the surface property with small metal having high electronegativity.

### TECHNICAL FIELD

The present invention relates to the field of chemistry, in particular, to the adsorbent for separating organochloride compound from liquid hydrocarbon and a process thereof.

### BACKGROUND OF THE INVENTION

The catalytic reforming is the chemical process used in the transformation of naphtha, which is obtained from the crude oil refining, having low octane value to have higher octane value. The product obtained from the catalytic reforming process is called reformates. The catalyst mostly used is platinum or rhenium on silica or silica-alumina composite support. Said catalyst is needed to be chlorinated in order to prevent the gathering of platinum or rhenium to larger particle which causes the deterioration of the catalyst.

However, the hydrogen gas produced from the reforming process would react with the chloride on the surface of the catalyst to form hydrogen chloride. The generated hydrogen chloride would react with the unsaturated hydrocarbon compounds to form organochloride compounds. Hydrogen chloride is a highly corrosive that can damage the equipment in process. Although the organochloride compounds are not as corrosive as hydrogen chloride, the organochloride compounds can be dissociated into hydrogen chloride at low temperature causing the corrosion.

The separation of hydrogen chloride and organochloride compounds from the feed stream can be performed by several methods. The method that has high efficiency and gives no effect on other hydrocarbon compounds in the feed stream is the adsorption process by subjecting the stream contaminated with hydrogen chloride and organochloride compounds to the fixed-bed adsorber containing adsorbent which is specific to said substance.

Normally, hydrogen chloride can be removed from the stream to remain the concentration less than 1 ppm by alumina with using alkaline metal as the promoter (as disclosed in U.S. Pat. No. 5,316,988). However, the removal of organochloride compounds is more difficult and there is limited data on adsorbent for organochloride compounds.

U.S. Pat. No. 3,862,900 discloses the process for removing organochloride compounds with 10X and 13X zeolites having pores in the range of 7 to 11 angstroms. It was found that the 13X zeolite had highest efficiency.

U.S. Pat. No. 8,551,328 B2 discloses that 13X zeolite having silicon to aluminium ratio lower than 1.25 gave better adsorption efficiency of organochloride compounds (vinyl chloride) than the standard 13X zeolite having silicon to aluminium ratio of 1.25.

U.S. Pat. No. 3,864,243 discloses the adsorption of organochloride compounds from hydrocarbon compounds using bauxite type alumina adsorbent calcined at the temperature in the range of 900-1,000° F. for 4-6 hours and then having porosity and high surface area. The adsorption efficiency of hydrocarbon compounds containing organochloride was 85-96% at room temperature and atmospheric pressure.

U.S. Pat. No. 5,107,061A discloses the adsorption of organochloride compounds which were 50-100 ppm of 2-butyl chloride and 5-10 ppm of t-butyl chloride from hydrocarbon compounds, exiting from the distillation column of polyisobutylene (PIB), which comprised 50% n-butane, 30% 1-butene, 15% 2-butene, 3% iso-butylene, and 2% isobutene. It was found that the adsorbent combination of 2 types which were alumina and NaX zeolite gave higher adsorption efficiency of organochloride compounds than using NaX zeolite alone.

Chinese patent no. 103611495A discloses the preparation of the adsorbent for organochloride compounds using three types of adsorbent which comprised: (1) X or Y zeolite having silicon to aluminium ratio in the range of 2-2.5 and having ion exchange with zinc (Zn); (2) macroporous inorganic material which was diatomaceous earth; and (3) clay, which was used to promote the strength, being bentonite and attapulgite. It was found that exchanging zinc ions in zeolite and adding inorganic material with suitable amount could significantly increase the adsorption efficiency of vinyl chloride when comparing with zeolite without ion exchange and without inorganic material added.

Arjang et al., 2018 studied the adsorption of organochloride compounds having starting concentration of 8.5-105 mg/L on the gamma-alumina support having specific surface area in the range of 230-400 m²/g and average particle size of 20 nm. It was found to give the adsorption efficiency up to 96% with the starting concentration of the organochloride compounds of 8.5 mg/L.

### SUMMARY OF INVENTION

The present invention relates to the adsorbent for separating organochloride compound from liquid hydrocarbon and a process thereof, wherein said adsorbent is the silica and aluminosilicate composite having infiltrate structure subjected to the modification of the surface property with small metal having high electronegativity.

### DESCRIPTION OF THE INVENTION

The present invention relates to the adsorbent for separating organochloride compound from liquid hydrocarbon and a process thereof, which will be described according to the following embodiments.

Any aspect being described herein also means to include the application to other aspects of this invention unless stated otherwise.

Technical terms or scientific terms used herein have definitions as understood by an ordinary person skilled in the art unless stated otherwise.

Any tools, equipment, methods, or chemicals named herein mean tools, equipment, methods, or chemicals being operated or used commonly by those person skilled in the art unless stated otherwise that they are tools, equipment, methods, or chemicals specific only in this invention.

Use of singular noun or singular pronoun with “comprising” in claims or specification means “one” and also include “one or more”, “at least one”, and “one or more than one”.

Hereafter, invention embodiments are shown without any purpose to limit any scope of the invention.

This invention relates to the adsorbent for separating organochloride compound from liquid hydrocarbon, wherein said adsorbent is the silica and aluminosilicate composite having infiltrate structure subjected to the modification of the surface property with small metal having high electronegativity.

In one aspect of the invention, the absorbent is the silica and aluminosilicate composite comprising small pores in the range of about 2 to 15 nm and large pores in the range of about 40 to 100 nm, wherein the ratio of the small pores to the large pores is from 0 to 1.

In one aspect of the invention, the silica and aluminosilicate composite have the ratio of silicon to aluminium in the range of 1-20, preferably in the range of 2-10.

In one aspect of the invention, the metal having high electronegativity is selected from zinc (Zn), iron (Fe), calcium (Ca), and magnesium (Mg), preferably zinc.

In one aspect of the invention, the adsorbent comprises the metal having high electronegativity in the range of about 0.1 to 10% by weight, preferably in the range of about 0.5 to 5% by weight.

In one aspect of the invention, the adsorbent comprises sodium metal in the range of 7 to 15% by weight.

In one aspect, said metal may be added into the silica and aluminosilicate composite adsorbent using commonly known method such as ion exchange or impregnation.

In one aspect, the silica and aluminosilicate composite adsorbent may be prepared using commonly known method and may be used in the form of powder, granule without subjected to forming process or subjected to forming process using binder selected from but not limited to alumina, silica, aluminosilicate, clay, or mixture thereof, or subjected to forming process without the use of binder.

In one aspect of the invention, the present invention relates to the process for separating organochloride compound from liquid hydrocarbon, comprising the step of contacting the liquid hydrocarbon mixed with the organochloride compound to the adsorbent in order to adsorb said organochloride compound and obtaining the liquid hydrocarbon having lower amount of organochloride compound, wherein said adsorbent is the silica and aluminosilicate composite having infiltrate structure subjected to the modification of the surface property with small metal having high electronegativity.

In one aspect of the invention, the adsorbent used in the process for separating according to the invention may be selected from the adsorbent as described above.

In one aspect of the invention, the organochloride compound is selected from alkyl chloride, allyl chloride, or mixture thereof. Preferably, the organochloride compound is selected from 1-chlorohexane, 1-chloro-2-methylbutane, 1-chloropentane, or mixture thereof, most preferably 1-chlorohexane.

In one aspect of the invention, the liquid hydrocarbon is the hydrocarbon having boiling point higher than 50° C. Preferably, the boiling point is in the range of about 50 to 210° C. The liquid hydrocarbon may be selected from toluene, paraffin, olefin, naphthene, aromatic, or mixture thereof.

In one aspect of the invention, the process for separating according to the invention is operated at the temperature of 30 to 50° C. and the pressure of atmospheric pressure to 10 bars.

In one aspect of the invention, the process according to the invention can separate the organochloride compound in liquid hydrocarbon, wherein the concentration of organochloride compound before contacting to the adsorbent is in the range of 2 to 200 ppm. After contacting to the absorbent giving the liquid hydrocarbon having lower amount of organochloride compound, the concentration of organochloride compound is less than 0.2 ppm.

In one aspect of the invention, the contacting of said liquid hydrocarbon containing organochloride compound to the adsorbent may be operated in the batch or continuous form, wherein the adsorbent may be used in the fixed bed system, moving bed system, or fluidized bed system, and may be used continuously in sequence or parallel.

The following examples are for demonstrating the embodiments of the invention, not for limiting the scope of the invention in any way.

To study the effect of the adsorbent on the separation efficiency of the organochloride compound from liquid hydrocarbon, 1-chlorohexane in toluene was used as an example of the organochloride compound in the liquid hydrocarbon without any purpose to limit the scope of the invention in any way.

### Preparation of the Adsorbent

### Preparation of the Silica and Aluminosilicate Composite

The step of the preparation of the silica and aluminosilicate composite having infiltrate structure was done by mixing sodium silicate solution or the solution that when being heated, gives oxide of silicon and aluminum hydroxide or the solution that when being heated, gives oxide of aluminum in water at the temperature about 30-70° C. The different ratios of silicon to aluminum are shown in table 1. Then, the pH was adjusted to 5.5-8.5 and the mixture was stirred for another 1 hour or more. After that, the pH was adjusted to 9-11 and the mixture was stirred for another 3-24 hours. The obtained gel was washed, dried at the temperature about 100-120° C. and calcined at the temperature about 500-700° C.

Treatment with Sodium (Na) Leaching

About 1 g of the silica and aluminosilicate composite prepared from method described above was dissolved in about 200 mL of deionized water and stirred at the temperature about 80° C. for about 30 min. This can be repeated as described above to obtain sodium content as desired. Then, the mixture was centrifuged. The obtained solid was dried at the temperature about 100° C. for about 12 hours. After that, the remaining organic substances were removed by calcination under atmospheric environment at the temperature about 630° C. for about 3 hours.

Treatment with Metal Having High Electronegativity

The silica and aluminosilicate composite having infiltrate structure or the silica and aluminosilicate composite treated with Na leaching prepared from method described above was subjected to the modification of the surface property with metal having high electronegativity (in this case, zinc) at the amount designed in percentage by weight of different samples as shown in table 1 by impregnation method using metal salt solution selected from zinc nitrate, chloride, or acetate. Then, the mixture was dried at the temperature about 100° C. for about 12 hours. After that, the mixture was calcined at high temperature in order to remove the organic substances at the temperature about 400 to 550° C. for about 2-4 hours.

The adsorbent obtained from above method was analyzed to determine surface area and pore size by N₂-physisorption technique. The results are shown in table 2.

TABLE 1
Adsorbent of different samples

| Sample | Type of adsorbent |
|---|---|
| Comparative sample 1 | 13X zeolite (commercial) |
| Sample according to | Silica and aluminosilicate composite having |
| the invention 1 | infiltrate structure with the silicon to aluminium |
| | ratio of 2.4 |
| Sample according | Silica and aluminosilicate composite having |
| to the invention 2 | infiltrate structure with the silicon to aluminium |
| | ratio of 2.4, being treated by Na leaching to |
| | obtain 9% by weight of sodium |
| Sample according | Silica and aluminosilicate composite having |
| to the invention 3 | infiltrate structure with the silicon to aluminium |
| | ratio of 2.4, being treated by Na leaching to |
| | obtain 8.5% by weight of sodium |
| Sample according | Silica and aluminosilicate composite having |
| to the invention 4 | infiltrate structure with the silicon to aluminium |
| | ratio of 4 |
| Sample according | Silica and aluminosilicate composite having |
| to the invention 5 | infiltrate structure with the silicon to aluminium |
| | ratio of 6 |
| Sample according | Silica and aluminosilicate composite having |
| to the invention 6 | infiltrate structure with the silicon to aluminium |
| | ratio of 8 |
| Sample according | Silica and aluminosilicate composite having |
| to the invention 7 | infiltrate structure with the silicon to aluminium |
| | ratio of 10 |
| Sample according | Silica and aluminosilicate composite having |
| to the invention 8 | infiltrate structure with the silicon to aluminium |
| | ratio of 8, being treated with 1.5% |
| | by weight of zinc |
| Sample according | Silica and aluminosilicate composite having |
| to the invention 9 | infiltrate structure with the silicon to aluminium |
| | ratio of 8, being treated with 3% |
| | by weight of zinc |

TABLE 2
Total surface area, pore size, total pore
volume, pore volume of small pores, pore volume
of large pores, and ratio of small pore to large pore

| | | | | Pore | Pore | Ratio |
|---|---|---|---|---|---|---|
| | | | | volume | volume | of |
| | | | | of small | of large | small |
| | | | Total | pores | pores | pore |
| | Surface | Average | pore | during | during | to |
| | area | pore size | volume | 2-15 nm | 40-100 nm | large |
| Sample | (m²/g) | (nm) | (cm³/g) | (cm³/g) | (cm³/g) | pore |
| Comparative | 489 | 0.68 | 0.25 | — | — | — |
| sample 1 | | | | | | |
| Sample | 126 | 8.8, 50.2 | 0.69 | 0.18 | 0.51 | 0.35 |
| according to | | | | | | |
| the invention | | | | | | |
| 1 | | | | | | |
| Sample | 148 | 9.4, 50.2 | 0.72 | 0.22 | 0.50 | 0.43 |
| according to | | | | | | |
| the invention | | | | | | |
| 2 | | | | | | |
| Sample | 141 | 8.8, 50.2 | 0.78 | 0.20 | 0.58 | 0.34 |
| according to | | | | | | |
| the invention | | | | | | |
| 3 | | | | | | |
| Sample | 113 | 6.3, 43.2 | 0.39 | 0.19 | 0.20 | 0.95 |
| according to | | | | | | |
| the invention | | | | | | |
| 4 | | | | | | |
| Sample | 135 | 3.6, | 0.46 | 0.18 | 0.28 | 0.65 |
| according to | | 8.1, 44.1 | | | | |
| the invention | | | | | | |
| 5 | | | | | | |
| Sample | 112 | 8.9, 43.2 | 0.66 | 0.19 | 0.47 | 0.41 |
| according to | | | | | | |
| the invention | | | | | | |
| 6 | | | | | | |
| Sample | 68 | 40.05 | 0.61 | — | 0.61 | 0 |
| according to | | | | | | |
| the invention | | | | | | |
| 7 | | | | | | |
| Sample | 64 | 65.82 | 0.52 | — | 0.52 | 0 |
| according to | | | | | | |
| the invention | | | | | | |
| 8 | | | | | | |

### Test of the Adsorption Efficiency for Adsorbent

Before being used, the adsorbent was dried in the oven to remove moisture at the temperature about 110° C. Then, the toluene containing 1-chlorohexane with the concentration of 1-chlorohexane in the range of 2 to 200 ppm was used to contact to about 1 g of the adsorbent for about 2 hours. The liquid phase was analyzed to determine the remaining 1-chlorohexane by gas chromatography equipped with electron capture detector (ECD). Then, the obtained results were used for the calculation to determine the adsorption efficiency and the amount of adsorbed 1-chlorohexane from the following equations. The results are shown in table 3.

$$Adsorption efficiency ( \% ) = \frac{\begin{matrix}amount of 1 \text{-} chlorohexane before adsorption - \\ amount of 1 \text{-} chlorohexane after adsorption\end{matrix}}{amount of 1 \text{-} chlorohexane before adsorption} \times 100$$

$$Amount of adsorbed substance i = \frac{\begin{matrix}amount of substance i before adsorption - \\ amount of substance i after adsorption\end{matrix}}{amount of adsorbent}$$

TABLE 3
Adsorption efficiency of 1-chlorohexane from toluene
at different starting concentrations

| | Adsorption efficiency |
|---|---|
| | of 1-chlorohexane (%) |
| Sample | Starting concentration 2 ppm |
| Comparative sample 1 | 4.60 |
| Sample according to the invention 1 | 4.49 |
| Sample according to the invention 2 | 5.67 |
| Sample according to the invention 3 | 19.54 |
| Sample according to the invention 4 | 5.67 |
| Sample according to the invention 5 | 6.26 |
| Sample according to the invention 6 | 6.43 |
| Sample according to the invention 7 | 6.79 |
| Sample according to the invention 8 | 7.20 |
| Sample according to the invention 9 | 9.80 |

The adsorption capability shown as the isotherm of the adsorption for each adsorbent was used to calculate the maximum adsorption by Langmuir isotherm equation. The results are shown in table 4.

TABLE 4
Maximum adsorption of 1-chlorohexane

| | Maximum adsorption (qₘₐₓ) |
|---|---|
| Sample | (μg/g of adsorbent) |
| Comparative sample 1 | 229 |
| Sample according to the invention 3 | 615 |
| Sample according to the invention 8 | 662 |

From all above, it can be said that the adsorbent according to the invention can effectively separate the organochloride compound from the liquid hydrocarbon as being stated in the objectives of this invention.

### BEST MODE OR PREFERRED EMBODIMENT OF THE INVENTION

Best mode or preferred embodiment of the invention is as provided in the description of the invention.

1. An adsorbent for separating organochloride compound from liquid hydrocarbon, wherein said adsorbent is a silica and aluminosilicate composite having infiltrate structure subjected to the modification of the surface property with small metal having high electronegativity;
wherein the silica and aluminosilicate composite comprises small pores in the range of 2 to 15 nm and large pores in the range of 40 to 100 nm, wherein the ratio of the small pores to the large pores is from 0 to 1.

2. (canceled)

3. The adsorbent according to claim 1, wherein the silica and aluminosilicate composite have the ratio of silicon to aluminium in the range of 2-10.

4. The adsorbent according to claim 1, wherein the metal having high electronegativity is selected from zinc (Zn), iron (Fe), calcium (Ca), and magnesium (Mg).

5. The adsorbent according to claim 4, wherein the metal having high electronegativity is zinc.

6. (canceled)

7. The adsorbent according to claim 1, wherein the adsorbent comprises the metal having high electronegativity in the range of 0.5 to 5% by weight.

8. The adsorbent according to claim 1, wherein the adsorbent comprises sodium metal in the range of 7 to 15% by weight.

9. A process for separating organochloride compound from liquid hydrocarbon, comprising the step of contacting the liquid hydrocarbon mixed with the organochloride compound to the adsorbent in order to adsorb said organochloride compound and obtaining the liquid hydrocarbon having lower amount of the organochloride compound, wherein said adsorbent is the silica and aluminosilicate composite having infiltrate structure subjected to the modification of the surface property with small metal having high electronegativity,
wherein the silica and aluminosilicate composite comprises small pores in the range of 2 to 15 nm and large pores in the range of 40 to 100 nm, wherein the ratio of the small pores to the large pores is from 0 to 1.

10. (canceled)

11. The process according to claim 9, wherein the silica and aluminosilicate composite have the ratio of silicon to aluminium in the range of 2-10.

12. The process according to claim 9, wherein the metal having high electronegativity is selected from zinc (Zn), iron (Fe), calcium (Ca), and magnesium (Mg).

13. The process according to claim 12, wherein the metal having high electronegativity is zinc.

14. (canceled)

15. The process according to claim 9, wherein the adsorbent comprises the metal having high electronegativity in the range of 0.5 to 5% by weight.

16. The process according to claim 9, wherein the adsorbent comprises sodium metal in the range of 7 to 15% by weight.

17. The process according to claim 9, wherein the organochloride compound is selected from alkyl chloride, allyl chloride, or mixture thereof.

18. (canceled)

19. (canceled)

20. The process according to claim 9, wherein the liquid hydrocarbon is the hydrocarbon having boiling point in the range of 50 to 210° C.

21. (canceled)

22. The process according to claim 9, wherein said process is operated at the temperature of 30 to 50° C. and the pressure of atmospheric pressure to 10 bars.

23. The process according to claim 9, wherein the liquid hydrocarbon having lower amount of the organochloride compound has the organochloride compound less than 0.2 ppm.

24-25. (canceled)
